(19) United States
(12) Patent Application Publication
(10) Pub. No.: US 2003/0013135 A1
(43) Pub. Date: Jan. 16, 2003

(54) In-cell NMR spectroscopy

(76) Inventor: Volker Doetsch, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/905,439

(22) Filed: Jul. 13, 2001

(51) Int. Cl. C12Q 1/68; G01N 33/567; G06F 19/00; G01N 33/48; G01N 33/50; G01N 24/00

(52) U.S. Cl. 435/7.21; 435/6; 702/20; 702/19; 436/173

(57) ABSTRACT

In-cell NMR procedure to enable one to observe protein conformations inside living cells. The signals produced by a single protein species can be distinguished using the method of the invention.

### STATEMENT OF GOVERNMENT RIGHTS

This invention was made with Government support under Grant (or Contract) No. GM56531-02, GM08284, MCB-9982596. The Government has certain rights in this invention.

### BACKGROUND OF THE INVENTION

Of all methods currently available for obtaining high resolution structures of biological macromolecules, NMR is the only one that can provide this information in solution under near physiological conditions (Kremer et al., Methods in Enzymology; James et al., Eds.; Academic Press: San Diego, 339:3-19 (2001); Stoll et al., Methods in Enzymology, 182:24-38 (1990)). Even NMR structures, however, are still determined in vitro. Often in vitro buffer conditions are not selected for their closest match to the natural environment of the protein, but to optimize experimental parameters such as solubility and sensitivity, or to minimize NMR buffer signals that could interfere with the signal from the analyte of interest.

A recent survey of buffer conditions used for NMR structure determinations showed that 27% of all structures were determined in unbuffered (or auto-buffered) solutions, 50% in phosphate buffer, 10% in acetate buffer and 9% in tris buffer (Hubbard et al., In 42nd ENC: Orlando, USA (2001)). Depending on the natural host cell and the exact cellular compartment, NMR buffer conditions can be substantially different from a protein's natural environment and may influence protein structure and dynamics. Furthermore, interactions with other cellular (macro-)molecules and post-translational modifications can alter the conformation of the protein.

NMR is also used for de novo structure determination of biologically relevant macromolecules. For example, NMR spectroscopy of proteins using techniques such as site-specific isotope labeling, yielded biologically relevant information on human hemoglobin (MW=65,000) as early as 1969 (Shulman et al., Science 165:251-257 (1969)), and subsequently also for significantly larger systems such as, for example, immunoglobulins (Arata et al. Methods in Enzymology 239:440-464 (1994)). Typically, however, NMR structural determination is available only for macromolecules of relatively small molecular sizes, generally below 30,000 Da (Wuthrich, K. (1996) NMR of Proteins and Nucleic Acids (Wiley, N.Y.); Wuthrich, K. (1995) NMR in Structure Biology (World Scientific, Singapore)).

In principle, NMR spectroscopy, as a non-invasive spectroscopic technique, is able to provide information about the structure and dynamics of biological macromolecules inside living cells. Indeed, in vivo NMR and magnetic resonance imaging are well established fields that use NMR spectroscopy to obtain information from living organisms ranging from cell suspensions to human beings (Li et al., NMR in Biomedicine, 9:141-155 (1996); Kanamori et al., Neurochemistry, 68:1209-1220 (1997); Bachert, P. Progress in Nuclear Magnetic Resonance Spectroscopy, 33:1-56 (1998); Spindler et al., J. Molecular and Cellular Cardiology, 31:2175-2189 (1999); Gillies, R. J., NMR in Physiology and Biomedicine; Academic Press: San Diego (1994)). Prior studies, however, have mainly focused on small molecules, which can be distinguished from all other molecules in the cell either because they are the most abundant or because they have been isotopically labeled.

NMR methods utilizing the labeling of proteins with NMR sensitive isotopes are known in the art. For example, one method for segmental isotopic labeling of proteins can be performed by the trans-splicing approach (Yamazaki et al., J. Am. Chem. Soc. 120:5591-5592 (1998)). Another preferred approach is “expressed protein ligation” in which synthetic peptides or recombinant proteins can be chemically ligated to the C terminus of peptides or recombinant proteins (Severinov et al., J. Biol. Chem. 273:16205-16209 (1998); Muir, et al., Proc. Natl. Acad. Sci. USA, 95:6705-6710 (1998), Xu et al., Proc. Natl. Acad. Sci., 96:388-393 (1999) and U.S. Pat. No: 09,191,890).

Moreover, the detection by NMR of specifically labeled compounds in cell lysates has been demonstrated (Gronenbom et al., Protein Science, 5:174-177 (1996)). Prior efforts have focused on the overexpression of proteins in ¹⁵N-labeled medium followed by cell lysis, buffer exchange to a suitable NMR buffer, and concentration of the protein resulted in virtually background free HSQC spectra, not on in vivo NMR of isotopically labeled proteins.

A strategy to obtain in-cell NMR spectra of NmerA expressed inside living E. coli bacteria and the first successful experiment with the small bacterial protein NmerA was published in a recent paper (Serber et al., J. Am. Chem. Soc., 123:2446-2447 (2001)). In addition, in-cell NMR spectra of osmoregulated glucans in the periplasm of Ralstonia solanacearum were recently reported (Lippens et al., In NMR in Supramolecular Chemistry; Pons, M., Ed.; Kluwer Academic Publishers, 191-226 (1999). These in-cell NMR experiments now open new avenues to characterize the conformation and dynamics of proteins and other biological macromolecules in their natural environment.

The relative orientations and motions of domains within many macromolecules are highly relevant to the biological activity of the macromolecule. For example, the orientations and motions of domains within proteins are key to the control of multivalent recognition, or the assembly of protein-based cellular machines. Therefore, it is not surprising that there has been a long and continuous need to determine the structures of biologically relevant macromolecules (e.g., nucleic acids and proteins), not only in their resting state, but also in their more dynamic state in their native environment. Data acquired from macromolecules in extracellular, in vitro investigations does not provide a complete understanding of the characteristics of the macromolecule in vivo.

There presently is a need for methods providing data relevant to the structure, dynamics and conformation of macromolecules in their native intracellular environment. Moreover, there is a need for a method to study the organization and interactions of a component of a selected macromolecule with other species (e.g., monitoring enzymatic reactions, DNA-protein interactions, ligand binding, and protein folding). Furthermore, there is a need to exploit such determinations in order to be able to design more potent drugs, pharmaceutical therapies and diagnostic agents.

Clearly, a method that provides rapid access to information about the physical state of intracellular macromolecules would be of great use in elucidating native macromolecule structure and drug-macromolecule interactions, among other applications. The present invention provides such a method.

### SUMMARY OF THE INVENTION

It has now been discovered that in cell NMR of intact, living cells provides structural, conformational and dynamic data for macromolecules within the cell. For example, the methods of the present invention can distinguish individual macromolecules, macromolecule conformations, and interactions of macromolecules with other species within an intact, living cell. In cell NMR spectroscopy provides a new tool for the characterization of macromolecules in their natural intracellular environment. In the invention described herein, different expression and labeling schemes are useful to optimize the sensitivity of the NMR measurements.

It has further been discovered that, contrary to general wisdom, growing the bacteria and expressing the protein in an isotopically enriched medium (e.g., ¹⁵N-labeled medium) does not result in the observation of hundreds of resonance lines from cellular components that become labeled with ¹⁵N as well (all the proteins, nucleic acids etc. become labeled, but they are at such a low concentration that they do not produce detectable signals). Surprisingly, however, only a very small number of background signals, mainly arising from ¹⁵N-incorporation into small molecules like amino acids, are detected using the in cell NMR acquisition method of the present invention. Even more surprisingly, growing the cells in ¹⁵N-labeled media prior to induction does not affect the amount of background signal significantly. Thus, the present invention provides a method in which background NMR signals from cellular components are not a limiting factor and high quality NMR spectra useful for analysis of structure and function of a macromolecule in a cell can be obtained under a wide variety of cell culture conditions.

The method of the present invention exploits the discovery that, even in living cells, only minimal background signals from non-specifically labeled cellular macromolecules are detected. In fact, the only background signals are derived from small molecules that are ¹⁵N-labeled.

Surprisingly, the methods allow NMR data to be collected and structural information to be extracted in the presence of large quantities of macromolecules that are typically removed as impurities for in vitro NMR analyses. It is further surprising that NMR spectra can be obtained with narrow enough line widths for extraction of structural information in the relatively high viscosity of the cell interior compared to the viscosities of solutions used for typical in vitro NMR experiments.

Other objects, advantages and aspects of the present invention will be apparent from review of the detailed description that follows and the claims appended hereto.

### BRIEF DESCRIPTION OF THE DRAWINGS

FIG. 1 is a comparison of in-cell HSQC spectra in the absence or presence of 35 μM rifampicin and 400 μM IPTG. All spectra were recorded with 4 scans per increment. (A) Induced bacteria without rifampicin. (B) Induced bacteria with rifampicin. (C) An in vitro HSQC of a purified NmerA sample. (D) Uninduced bacteria without rifampicin. (E) Uninduced bacteria with rifampicin.

FIG. 2 shows the influence of the bacterial growth protocol on the quality of the resulting NMR spectra. (A) In-cell HSQC of an NmerA sample. The same ¹⁵N-labeled minimal medium was used to grow the bacteria to an optical density of 0.8 and for expressing the protein following induction with 0.4 mM IPTG. (B) The bacteria were harvested after reaching an optical density of 0.8 in ¹⁵N-labeled minimal medium by centrifugation and were resuspended in fresh ¹⁵N-labeled minimal medium followed by induction with IPTG. (C) The cells were grown in unlabeled LB medium, harvested by centrifugation and resuspended in ¹⁵N-labeled minimal medium for protein expression. In all three cases the bacteria were harvested 4 hours after induction.

FIG. 3 is in-cell HSQC spectra of NmerA collected after varying times following induction of protein expression on ¹⁵N-labeled minimal medium. (A) HSQC spectrum recorded after 10 minutes, (B) after 30 minutes, (C) after one hour and (D) after 2 hours of induction. One-dimensional cross-section taken at the position indicated by the dotted line is shown as well.

FIG. 4 is a12% SDS-polyacrylamide gel of 2 μl samples taken from the NMR samples of FIG. 3. The letters correspond to the letters of the HSQC spectra. A molecular weight marker is shown at the left hand side. The arrow marks the location of the NmerA band.

FIG. 5 is a comparison of the quality of in-cell NMR spectra of NmerA, which were obtained by protein expression in (A) ¹⁵N-labeled minimal medium and (B) 98% ¹⁵N-labeled, 97% deuterated rich medium (Celtone-dN, Martek). In both cases the samples were grown in unlabeled LB medium before they were transferred to the labeled media for protein expression. One-dimensional cross sections taken along the acquisition dimensions at the position indicated by the dotted line are shown on top of both spectra.

FIG. 6 is the in cell HSQC-spectra of selectively ¹⁵N-lysine labeled NmerA (A) and human calmodulin (B). The calmodulin spectrum was measured with 16 scans per increment.

### DETAILED DESCRIPTION OF THE INVENTION AND THE PREFERRED EMBODIMENTS

Introduction

Knowledge of the detailed three-dimensional structure of any given macromolecule is critical for developing drugs that regulate or otherwise alter the behavior of the macromolecule (e.g., a protein that is malfunctioning in a metabolic pathway). Currently, there are two major strategies for determining the detailed three-dimensional structure of a macromolecule: X-ray crystallography and nuclear magnetic resonance. X-ray crystallographic analysis of macromolecules requires the time-consuming process of preparing high quality crystals, whereas classical NMR three-dimensional analysis of macromolecules is typically performed using purified molecules in solution.

The NMR spectra of macromolecules inside living cells differs from those obtained using in vitro macromolecule NMR experiments in several ways. For example, instead of dissolving the protein in a homogeneous aqueous buffer solution, macromolecules inside living cells are in an inhomogeneous environment that contains hundreds of different protein species, nucleic acids, lipids and a huge arsenal of small molecules. The greatest obstacle for in cell NMR experiments is to selectively distinguish the macromolecule's resonances from the resonances of all other molecules inside the cell.

The present invention overcomes many of the difficulties discussed above and provides for the first time an in-cell NMR method that allows the characterization of a single macromolecule in an intact, living cell.

Definitions

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization described below are those well known and commonly employed in the art. Standard techniques are used for nucleic acid and peptide synthesis. The techniques and procedures are generally performed according to conventional methods in the art and various general references (see generally, Sambrook et al MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well known and commonly employed in the art. Standard techniques, or modifications thereof, are used for chemical syntheses and chemical analyses.

As used herein, “nucleic acid” means DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, points of attachment and fluxionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications such as capping with a fluorophore (e.g., quantum dot) or another moiety.

As used herein a “macromolecule” is a structured molecule that contains one or more components and has a molecular weight of at least about 1000 daltons. Macromolecules of the present invention include biopolymers; synthetic chemical polymers; and chimeric polymers as defined below. A macromolecule of the invention can include one or more post translational modifications including, for example, glycosylation, phosphorylation, lipidation, ubiquitination or farnesylation.

As used herein a “biopolymer” is a polymer of monomeric units or derivatives thereof, which are naturally found in living cells. Examples of biopolymers include, but are not limited to saccharide polymers; amino acid polymers including, but not limited to, proteins, enzymes, antibodies, and receptors, and peptides comprising an unnatural amino acid constituent; glycopeptides; and nucleic acid polymers including mRNAs, cDNAs, and nucleic acids comprising nucleotide analogs.

As used herein a “chimeric polymer” is a macromolecule, which comprises multiple monomeric units (or derivatives thereof) and is not naturally made e.g., as opposed to a macromolecule that is a product of nature. A chimeric polymer can be a polymer comprising a biopolymer or fragment thereof and a synthetic chemical polymer. A particular type of chimeric polymer is a chimeric protein as defined below.

As used herein the terms “chimeric protein” or “chimeric peptide” are used interchangeably with the terms “fusion protein” and “fusion peptide” respectively and are amino acid polymers that do not naturally exist in nature but comprise at least a portion of one or more naturally occurring proteins or peptides.

“Peptide” refers to a polymer in which the monomers are amino acids and are joined together through amide bonds, alternatively referred to as a “polypeptide.” Unnatural amino acids, for example, β-alanine, phenylglycine and homoarginine are also included under this definition. Amino acids that are not gene-encoded may also be used in the present invention. Furthermore, amino acids that have been modified to include reactive groups may also be used in the invention. All of the amino acids used in the present invention may be either the D - or L-isomer. The L-isomers are generally preferred. In addition, other peptidomimetics are also useful in the present invention. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid but, which function in a manner similar to a naturally occurring amino acid.

“Antibody,” as used herein, generally refers to a polypeptide comprising a framework region from an immunoglobulin or fragments or immunoconjugates thereof that specifically binds and recognizes an antigen. The recognized immunoglobulins include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The term “drug” or “pharmaceutical agent,” refers to bioactive compounds that affect an organism. Moreover, the terms also encompass drugs in a prodrug form. Prodrugs are those compounds that readily undergo chemical changes under physiological conditions to provide the compounds of interest in the present invention.

The term “candidate drug,” refers to a drug, pharmacophore or chemotype that is under investigation as a potential therapeutic agent.

As used herein, a “biological compartment” is a naturally occurring chamber, or derivative thereof, having an interior space confined by a membrane or wall such that a macromolecule in the interior space is prevented from being released to the external space. The term can include, for example, a cell virus or subcellular organelle such as a mitochondria, chloroplast, golgi body, vesicle, vacuole, nucleus, or endoplasmic reticulum. The term can include a derivative of a naturally occurring chamber so long as the chamber retains a membrane or wall sufficient to prevent release of a macromolecule to the exterior space. A modified chamber can include, for example, a protoplast or organelle from which components have been removed or added or a microsome formed from a native cell such as a liver microsome formed from a liver cell.

A “living cell,” as used herein, refers to a cell carrying out metabolic or other function sufficient to preserve or replicate its genomic DNA. A living cell can be identified by well known methods in the art including, for example, presence of an intact membrane, staining by a particular dye, ability to produce progeny or, in the case of a gamete, ability to combine with a second gamete to produce a viable offspring. Cells useful in the invention include prokaryotic and eukaroytic cells. Prokaryotic cells include bacteria such as E. coli. Eukaryotic cells include yeast cells and cells derived from plants and animals, for example mammalian, insect (e.g., spodoptera) and particularly human cells. Cells are particularly useful when they are naturally nonadherent or have been treated not to adhere to surfaces, for example by trypsinization. Cells grown in cell and tissue culture are also useful in the invention.

As used herein the term “intact,” when used in reference to a biological compartment, is a compartment where the membrane or wall contains the contents of the compartment, thereby preventing the release of interior contents to the exterior of the compartment. An intact cell can further have an intact periplasmic membrane. Containment of the compartment contents need not be absolutely complete, but can be substantially complete, such that the macromolecule of interests is still contained within the compartment in substantially the same environment as a naturally occurring compartment in its normally metabolic state.

As used herein the term “structural information” refers to a representation of a conformation of a macromolecule in whole or in part at a resolution sufficient to determine the relative locations of two or more atoms. The term can include, for example, a representation that can be used to determine the relative position of two or more atoms within less than 10 Å, less than 5 Å, less than 3 Å, less than 2.5 Å or less than 2 Å.

As used herein the term “radiofrequency energy” refers to radiation having an energy sufficient to produce an excited nucleus that is NMR detectable. The term can include radiation having a frequency of at least about 50 MHz, 100 MHz, 300 MHz, 500 MHz, 1 GHz, 2 GHz or higher.

The Method

In a first aspect, the present invention provides a method of collecting a NMR data set for a selected macromolecule in an intact, living cell. The macromolecule is labeled with an NMR-detectable nucleus, such that the nucleus is present in the macromolecule in an amount greater than is naturally abundant in the macromolecule. Any NMR-detectable nucleus is useful in the present invention, such as ¹H, ¹⁵N, ¹³C, ¹⁹F, ³¹P and combinations thereof.

The method of the invention includes contacting the cell with radio frequency energy in an NMR experiment. Upon contacting the cell with the radio frequency energy the NMR-detectable nucleus is excited. Following the excitation of the NMR-detectable nucleus, radio frequency data is collected from the excited NMR-detectable nucleus. The radio frequency data that is collected is used to assemble an NMR data set, which is preferably further processed to extract structural information for the selected macromolecule from the data set.

The methods of the present invention can be performed on any macromolecule that is amenable to NMR spectroscopic analysis. In a preferred embodiment the macromolecule is a biopolymer. In a particular embodiment the biopolymer is a peptide. In another embodiment the biopolymer is a protein (including glycoproteins, lipoproteins and chimeric proteins) such as an enzyme, a transcription factor and/or DNA binding protein, an antibody, a cytokine, a receptor, a ligand for a receptor, or a structural protein. In yet another embodiment the biopolymer is a carbohydrate. In a related embodiment the biopolymer is a lipopolysaccharide. In still another embodiment the biopolymer is a nucleic acid (e.g., DNA, mRNA, ribosomal RNA, tRNA or ribozyme). The macromolecule of the present invention can also be a chimeric polymer formed between two or more biopolymers or a biopolymer and a synthetic chemical polymer. The selected components of the macromolecules of the present invention include protein domains, and prosthetic groups (e.g., lipids, lipid polysaccharides as well as small organic molecules such as flavins, porphyrins and the like).

As discussed in the previous section, the macromolecule can be labeled with an NMR-detectable nucleus, such as ¹⁵N, using any means well known in the art. In an exemplary embodiment, the macromolecule is prepared in recombinant form using transformed host cells. Any labeled macromolecule that gives a high resolution NMR spectrum and can be partially or uniformly labeled with ¹⁵N can be used. In a preferred embodiment, the macromolecule is a polypeptide. The preparation of an exemplary uniformly ¹⁵N-labeled polypeptide macromolecules is set forth hereinafter in the Examples.

Those of skill in the art are aware of NMR acquisition sequences that are of general utility in practicing the present invention. In practice, the perturbed atoms in large molecules can be identified using a multidimensional multinuclear NMR method to identify NMR cross-peaks corresponding to the perturbed atoms. Heteronuclear NMR experiments are particularly useful with larger proteins as described in Cavanaugh et al., PROTEIN NMR SPECTROSCOPY: PRINCIPLES AND PRACTICE, Ch. 7, Academic Press, San Diego, Calif. (1996) For example, two dimensional NMR experiments can measure the chemical shifts of two types of nuclei. A well established 2-D method is the ¹H-¹⁵N heteronuclear single quantum coherence (HSQC) experiment. Another method is the heteronuclear multiple quantum coherence (HMQC) experiment. Numerous other variant experiments and modifications are known in the art including nuclear Overhauser enhancement spectroscopy experiments (NOESY), for example NOE experiments involving a {¹H, ¹H} NOESY step.

Higher-dimensional NMR experiments can be used to measure the chemical shifts of additional types of nuclei and to eliminate problems with cross peak overlap if spectra are too crowded. In particular, the NMR method used can correlate ¹H, ¹³C, and ¹⁵N (Kay et al., J. Magn. Reson., 89:496-514 (1990); Grzesiek and Bax, J. Magn. Reson., 96:432-440 (1992)), for example in an HNCA experiment. Other heteronuclear NMR experiments can be used so long as the transfer of magnetization to all CL and protein protons is only to or from amide protons on the protein, since all carbon-attached protons in the protein are replaced with deuterons. Such experiments include HNCO, HN(CO)CA, HN(CA)CO, and CBCA(CO)NH experiments.

Preferred sequences are those used in triple resonance NMR spectroscopy (Kay et al., J. Magn. Reson. 89:495-514 (1990); Inouye, U.S. Pat. No. 6,162,627; Fesik, U.S. Pat. No. 5,989,827; and Piotto et al., U.S. Pat. No. 5,475,308). Presently preferred NMR pulse sequences include, but are not limited to, those sequences associated with HSQC and TROSY (“transverse relaxation-optimized spectroscopy” (Pervushin, U.S. Pat. No. 6,133,736)) experiments and hybrids and modifications thereof. A pulse sequence to be used in the methods of the invention can be selected according to a variety of well known criteria, including, for example, size of the macromolecule to be analyzed. Thus, the methods of the invention can be used to determine structural information for a macromolecule that is at least 5 kDa, at least 10 kDa, or at least 10 kDa. Pulse sequences designed for higher molecular weight macromolecules such as TROSY can be used in the methods of the invention for macromolecules including, for example, those that are at least 30 kDa, at least 35 kDa or higher (see, e.g., Cover et al, J. Magn. Reson. 151: 60 (2001); Fernandez et al, Proc Natl. Acad. Sci. USA 98: 2358 (2001); and Peruvshin, U.S. Pat. No. 6,133,736). Higher molecular weight macromolecules can be used in the methods by using other pulse sequences known in the art including, for example, SEA-TROSY. Thus, the methods can be used to obtain structural and functional information for macromolecules having molecular weights of at least 50 kDa, at least 70 kDa, or at least 170 kDa.

In a preferred embodiment of the present invention, the cell is present in the sample as an aqueous dispersion, such as a slurry. A factor that can change the cytoplasmic environment is the high cellular density in the NMR tube. This high density leads to oxygen starvation for the bacteria, switching them to an anaerobic state, which changes the metabolism of the bacteria and influences the intracellular pH. Modified NMR tubes or bioreactors for the NMR experiments can be used to exchange media and provide the bacteria with oxygen. Several different designs for these bioreactors have already been used for in vivo spectroscopy with small molecules (Egan, W. M. The use of perfusion systems for nuclear magnetic resonance studies in cells.; CRC Press: Boca Raton, Fla., 1987; Vol. 1; Szwergold, B. S. Annual Review of Physiology, 54:775-798 (1992); and Cohen et al., Monitoring intracellular metabolism by nuclear magnetic resonance.; Academic Press: San Diego, 1989; Vol. 177).

In a preferred embodiment, the cell density in the slurry is selected such that it provides an optimum between maximizing the signal intensity and obtaining a reasonable linewidth. Even more preferably, a density is selected such that, a uniform cell distribution can be maintained for several hours with only little sedimentation. In a still further preferred embodiment, a slurry having from about 20% to about 30% cell density is used.

The large water signal produced during in cell NMR studies is preferably suppressed by spoiling gradients.

To facilitate processing of the NMR data, computer programs are used to transfer and automatically process the multiple two-dimensional NMR data sets, including a routine to automatically phase the two-dimensional NMR data. The analysis of the data can be facilitated by formatting the data so that the individual HSQC spectra are rapidly viewed and compared to the HSQC spectrum of the control sample containing only the vehicle for the added compound (DMSO), but no added compound. Detailed descriptions of means of generating such two-dimensional ¹⁵N/¹ H correlation spectra are set forth hereinafter in the Examples.

The methods of the invention can, therefore, be used to determine structural information for a macromolecule in a cell under conditions that are different from those normally employed for structural analysis. Specifically, typical NMR methods of structure analysis for macromolecules are performed in vitro using highly purified samples in relatively low viscosity solutions. In contrast, the methods of the invention provide the advantage of being able to collect an NMR data set and extract structural information for a macromolecule in the presence of high concentrations of other biological components compared to those normally present in an in vitro NMR analysis. For example, E. coli can contain concentrations of protein, RNA, and DNA in the ranges of 200-320 mg/ml, 75-120 mg/ml., and 11-18 mg/ml, respectively (Elowitz et al., J. Bact., 181:197-203 (1999)). Thus, the methods can be used for structural analysis of a macromolecule in a cell in the presence of at least 200 mg/ml protein, at least 250 mg/ml protein, or at least 300 mg/ml protein.

The method of the present invention is practiced using cells in which macromolecules enriched in the amount of a low natural abundance NMR-detectable nucleus (“labeled”) are expressed. Substantially any method known to those of skill in the art can be used to produce cells useful in practicing the present invention. In a preferred embodiment of the present invention, a labeled cell is prepared by a method, which includes transforming an unlabeled precursor of the labeled cell with a nucleic acid encoding the selected macromolecule. The nucleic acid is preferably operably linked to a promoter, which is non-native to the unlabeled precursor cell (e.g., a phage promoter in a bacterium). The transformed cell is then incubated in a medium that included the NMR-detectable nucleus. Prior to, concurrent with, and/or following the incubation, the cell is induced to begin synthesis of the labeled macromolecule to produce the labeled cell.

In another embodiment, the method of preparing the labeled cell further includes inhibiting essentially all transcription in the transformed cell, which is under control of promoters native to the unlabeled precursor cell (see, Example 3). Although the transcription under the control of the native promoters is retarded, the transcription under control of the non-native promoter proceeds, preferably unretarded.

The method of overexpression and labeling set forth herein provides the advantage of increasing the resolution signals arising from nuclear resonances associated with species inside a biological compartment.

The medium in which the cells are grown is generally an art recognized medium useful in growing the cell selected for labeling and investigation using the methods of the invention. In addition to the normal ingredients, media useful in preparing cells to be used in the present method will also include a labeled species, which is typically a precursor of the macromolecule of interest. The nature of the labeled species in the medium, which is labeled with the NMR sensitive nucleus is dependent upon the nature of the macromolecule of interest. For example, when the macromolecule is a saccharide, the labeled species in the medium can include a labeled saccharide nucleotide, or other saccharide precursor. In a preferred embodiment, in which the macromolecule is a polypeptide, the medium includes an amino acid or amino acid precursor labeled with the NMR sensitive nucleus.

In another embodiment, the media includes one or more additional NMR-detectable nucleus. When more than one NMR-detectable nucleus is used in the media, one of the nuclei is preferably deuterium. The deuterium may be present as an exchangeable deuterium associated with a macromolecule precursor, or it may be present in the media in the form of a solvent (e.g., D₂O, d₆-DMSO, DCl, etc.).

Macromolecule Interactions With Other Species

Another aspect of the present invention is a method of identifying an agent (e.g., a drug) that interacts with a selected macromolecule in a cell. In an exemplary embodiment, the species interacting with the macromolecule affects the orientation or chemical shifts of the nuclei located proximate the site of interaction.

In another embodiment. the invention provides a method for measuring the relaxation rate of a heteronucleus contained by a selected component of a macromolecule, in solution, in the presence of a drug. The relaxation rate of the heteronucleus in the absence of the drug is also measured under otherwise the same conditions. In a preferred embodiment, the overall hydrodynamic characteristics and the local internuclear vector orientation of the selected components of the macromolecule are derived and it is determined whether there is a change in orientation, chemical shift or other relevant NMR detectable parameter of the selected components of the macromolecule in the presence of the drug. When a change in a detectable parameter is determined, the agent is identified as capable of affecting a property of selected components of the macromolecule in vivo.

The methods disclosed herein can be used to determine heretofore unknown binding sites in biologically relevant macromolecules (e.g., proteins and nucleic acids) for rational drug design and/or development of diagnostic agents, or as an aid in the selection of optimized antisense molecules and/or gene therapy reagents. Thus, the use of the structural determinations uniquely enabled by the present invention provides a means for identifying agents that can interact with macromolecules that can act as drugs, diagnostic agents, and the like. Furthermore, such methodology allows the refinement of the structures of such agents to optimize their properties through further defining the basis of the binding of the agent to the macromolecule.

As discussed above, any macromolecule labeled with an NMR-detectable nucleus can be used in the methods of the present invention. Because of the importance of cellular polypeptides in medicinal chemistry, a preferred macromolecule is an intracellular polypeptide. The use of the present invention to investigate the interaction between an intracellular macromolecule and an exogenously administered species, such as a drug is exemplified herein by reference to a peptide as a representative macromolecule. The focus of the discussion on polypeptides is for clarity of illustration and should not be construed to limit the scope of the intermolecular interactions that the present method is useful at elucidating.

As discussed in the previous section, an intracellular polypeptide can be labeled with an NMR-detectable nucleus, such as ¹⁵N using any means well known in the art. In a preferred embodiment, the macromolecule is prepared in recombinant form using transformed host cells. Any polypeptide that gives a high resolution NMR spectrum and can be partially or uniformly labeled with ¹⁵N can be used. The preparation of an exemplary uniformly ¹⁵N -labeled polypeptide macromolecules is set forth in the Examples.

In one embodiment the macromolecule is a protein and the agent identified is a potential agonist or antagonist of the protein. In either case, depending on the identity of the protein, the potential agonist or antagonist can be further characterized by biochemical assays, for example, that measure an activity of the protein. In a particular embodiment of this type, the protein is a multi-domain protein.

In another embodiment the macromolecule comprises a DNA binding protein bound to its nucleic acid binding site, and the drug identified is a potential agonist or antagonist of the DNA binding protein-nucleic acid interaction. Again, in either case, depending on the identity of the DNA binding protein, the potential agonist or antagonist can be further characterized by biochemical assays, for example, that measure an aspect of the DNA binding protein-nucleic acid interaction, such as an affinity constant.

There are numerous advantages to the NMR-based discovery and design processes of the present invention. First, because a process of the present invention identifies ligands by directly measuring the structure of the ligand and/or macromolecule when bound together, the problem of false positives is significantly reduced. Moreover, because the present process identifies specific binding sites on the macromolecule, the problem of false positives resulting from the non-specific binding of compounds to the macromolecule at high concentrations is eliminated.

Second, the problem of false negatives is significantly reduced because the present process can identify compounds that specifically bind to the macromolecule with a wide range of dissociation constants. The dissociation or binding constant for compounds can also be determined with the present process.

Because the location of the bound ligand can be determined from an analysis of the chemical shifts of the macromolecule that change upon the addition of the ligand and from nuclear Overhauser effects (NOEs) between the ligand and biomolecule, the binding of a second ligand can be measured in the presence of a first ligand that is already bound to the macromolecule. The ability to simultaneously identify binding sites of different ligands allows a skilled artisan to: a) define negative and positive cooperative binding between ligands; and b) design new drugs by linking two or more ligands into a single compound while maintaining a proper orientation of the ligands to one another and to their binding sites.

Further, if multiple binding sites exist on the macromolecule, the relative affinity of individual binding moieties for the different binding sites can be measured from an analysis of the chemical shift changes of the macromolecule as a function of the added concentration of the ligand. By simultaneously screening numerous structural analogs of a given compound, detailed structure/activity relationships about ligands is provided.

The NMR methods set forth herein are also useful in conjunction with computer modeling using a docking program such as GRAM, DOCK, or AUTODOCK (Dunbrack et al., Folding & Design 2:27-42 (1997)). The modeling procedure can include computer fitting of potential drugs to a particular macromolecule to ascertain how well the shape and the chemical structure of the potential ligand will complement or interfere with the in vivo structure of the macromolecule determined by the present NMR method (Bugg et al., Scientific American, Dec.: 92-98 (1993); West et al., TIPS, 16:67-74 (1995)). Computer programs can also be employed to estimate the attraction, repulsion, and steric hindrance of the potential drug to a binding site, for example. Generally the tighter the fit (e.g., the lower the steric hindrance, and/or the greater the attractive force), the more potent the drug will be since these properties are consistent with a tighter binding constant. Furthermore, the more specificity in the design of a potential drug the more likely that the drug will not interfere with related proteins. This will minimize potential side effects due to unwanted interactions with other proteins.

The structural analysis disclosed herein in conjunction with computer modeling allows the selection of a finite number of rational chemical modifications, as opposed to the countless number of essentially random chemical modifications that could be made, any of which might lead to a useful drug. Each chemical modification requires additional chemical steps, which while being reasonable for the synthesis of a finite number of compounds, quickly becomes overwhelming if all possible modifications needed to be synthesized. Thus, through the use of the NMR methodology disclosed herein in conjunction with computer modeling, a large number of these compounds can be rapidly screened on the computer monitor screen, and a few likely candidates can be determined without the laborious synthesis of untold numbers of compounds; the de novo synthesis of one or even a relatively small group of specific compounds is reasonable in the art of drug design.

Once a potential drug (e.g., agonist or antagonist) is identified it can then be tested in any standard assay for the macromolecule depending of course on the macromolecule, including in high throughput assays. When a suitable potential drug is identified, a further NMR structural analysis can optionally be performed to determine the three dimensional structure of the agent. Computer programs that can be used to aid in solving the three-dimensional structure include QUANTA, CHARMM, INSIGHT, SYBYL, MACROMODEL, and ICM, MOLMOL, RASMOL, and GRASP (Kraulis, J. Appl Crystallogr., 24:946-950 (1991)).

Moreover, as the spectra of use in the present method can be rapidly obtained, it is feasible to screen a large number of compounds (Shuker et al., Science, 274:1531-1534 (1996)) by, for example, ¹⁵N-HSQC. Thus, the present method is of use in NMR-based high throughput screening of compounds.

In another embodiment, two or more compounds are screened for binding to two nearby sites on a macromolecule. In this case, a compound that binds a first site of the macromolecule does not bind a second nearby site. Binding to the second site can be determined, for example, by monitoring changes in a different set of amide chemical shifts in either the original screen or a second screen conducted in the presence of a ligand (or potential ligand) for the first site. From an analysis of the chemical shift changes the approximate location of a potential ligand for the second site is identified.

The present method also provides a process for determining the dissociation constant between a macromolecule and a ligand that binds to that macromolecule. In a preferred embodiment, the process includes generating a first two-dimensional ¹⁵N/¹H NMR correlation spectrum of a ¹⁵N-labeled macromolecule in a cell. The cell containing the labeled macromolecule is then titrated with various concentrations of a ligand. A two-dimensional ¹⁵N/¹H NMR correlation spectrum is generated at each concentration of ligand during the titration. The spectra from each step of the titration are compared to each other and to the first spectrum to quantify differences in those spectra as a function of changes in ligand concentration. The differences are used to calculate the dissociation constant (K_{D}) for the macromolecule-ligand complex.

Informatics

As high-resolution, high-sensitivity datasets acquired using the methods of the invention become available to the art, significant progress in the areas of diagnostics, therapeutics, drug development, toxicology, biosensor development, and other related areas will occur. For example, disease markers can be identified and utilized for better confirmation of a disease condition or stage (see, U.S. Pat. No. 5,672,480; 5,599,677; 5,939,533; and 5,710,007). Subcellular toxicological information can be generated to better direct drug structure and activity correlation (see, Anderson, L., “Pharmaceutical Proteomics: Targets, Mechanism, and Function,” paper presented at the IBC Proteomics conference, Coronado, Calif. (Jun. 11-12, 1998)). Subcellular toxicological information can also be utilized in a biological sensor device to predict the likely toxicological effect of chemical exposures and likely tolerable exposure thresholds (see, U.S. Pat. No. 5,811,231).

Thus, in another preferred embodiment, the present invention provides a database that includes at least one set of NMR assay data. The data contained in the database is acquired using a method of the invention. The database can be in substantially any form in which data can be maintained and transmitted, but is preferably an electronic database. The electronic database of the invention can be maintained on any electronic device allowing for the storage of and access to the database, such as a personal computer, but is preferably distributed on a wide area network, such as the World Wide Web.

The methods described herein for determining in vivo structural, conformational and dynamic data for a variety of macromolecular species from a biological sample provide an abundance of information, which can be correlated with pathological conditions, predisposition to disease, drug testing, therapeutic monitoring, gene-disease causal linkages, identification of correlates of immunity and physiological status, among others. Although the data generated from the method of the invention is suited for manual review and analysis, in a preferred embodiment, prior data processing using high-speed computers is utilized.

An array of methods for indexing and retrieving biomolecular information is known in the art. For example, U.S. Pat. Nos. 6,023,659 and 5,966,712 disclose a relational database system for storing biomolecular sequence information in a manner that allows sequences to be catalogued and searched according to one or more protein function hierarchies. U.S. Pat. No. 5,953,727 discloses a relational database having sequence records containing information in a format that allows a collection of partial-length DNA sequences to be catalogued and searched according to association with one or more sequencing projects for obtaining full-length sequences from the collection of partial length sequences. U.S. Pat. No. 5,706,498 discloses a gene database retrieval system for making a retrieval of a gene sequence similar to a sequence data item in a gene database based on the degree of similarity between a key sequence and a target sequence. U.S. Pat. No. 5,538,897 discloses a method using mass spectroscopy fragmentation patterns of peptides to identify amino acid sequences in computer databases by comparison of predicted mass spectra with experimentally-derived mass spectra using a closeness-of-fit measure. U.S. Pat. No. 5,926,818 discloses a multi-dimensional database comprising a functionality for multi-dimensional data analysis described as on-line analytical processing (OLAP), which entails the consolidation of projected and actual data according to more than one consolidation path or dimension. U.S. Pat. No. 5,295,261 reports a hybrid database structure in which the fields of each database record are divided into two classes, navigational and informational data, with navigational fields stored in a hierarchical topological map which can be viewed as a tree structure or as the merger of two or more such tree structures. Algorithms which can be used to compare structures are known in the art and include, for example, CATALYST (Molecular Simulations, Inc., San Diego, Calif.), PRIZM, and THREEDOM, which is part of the INTERCHEM package which makes use of an Icosahedral Matching Algorithm (Bladon, J. Mol. Graphics, 7:130 (1989)) for the comparison and alignment of structures.

The present invention provides a computer database, which includes a computer and software for storing in computer-retrievable form assay data records cross-tabulated, for example, with data specifying the source of the macromolecule-containing sample from which each data record was obtained.

In an exemplary embodiment, at least one of the sources of macromolecule-containing sample is from a tissue sample known to be free of pathological disorders. In a variation, at least one of the sources is a known pathological tissue specimen, for example, a neoplastic lesion or a tissue specimen containing a pathogen such as a virus, bacteria or the like. In another variation, the assay records cross-tabulate one or more of the following parameters for each target species in a sample: (1) a unique identification code, which can include, for example, a macromolecule molecular structure and/or characteristic NMR coordinate; (2) sample source; and (3) absolute and/or relative measure of an in vivo property of the macromolecule present in the cell.

The invention also provides for the storage and retrieval of a collection of target data in a computer data storage apparatus, which can include magnetic disks, optical disks, magneto-optical disks, DRAM, SRAM, SGRAM, SDRAM, RDRAM, DDR RAM, magnetic bubble memory devices, and other data storage devices, including CPU registers and on-CPU data storage arrays. Typically, the target data records are stored as a bit pattern in an array of magnetic domains on a magnetizable medium or as an array of charge states or transistor gate states, such as an array of cells in a DRAM device (e.g., each cell comprised of a transistor and a charge storage area, which may be on the transistor). In one embodiment, the invention provides such storage devices, and computer systems built therewith, comprising a bit pattern encoding a NMR data record comprising unique identifiers for at least 10 target data records cross-tabulated with target source.

When the macromolecule is a peptide or nucleic acid, for example, the invention preferably provides a method for identifying related peptide- or nucleic acid-derived data, comprising performing a computerized comparison between the peptide- or nucleic acid-derived data record stored in or retrieved from a computer storage device or database and at least one other sequence. The comparison can include, for example, a sequence analysis or comparison algorithm or computer program embodiment thereof (e.g., FASTA, TFASTA, GAP, BESTFIT) and/or the comparison may be of the relative amount of a peptide or nucleic acid sequence in a pool of sequences determined from a polypeptide or nucleic acid sample of a specimen.

The invention also preferably provides a magnetic disk, such as an IBM-compatible (DOS, Windows, Windows95/98/2000, Windows NT, OS/2) or other format (e.g., Linux, SunOS, Solaris, AIX, SCO Unix, VMS, MV, Macintosh, etc.) floppy diskette or hard (fixed, Winchester) disk drive, comprising a bit pattern encoding data from an NMR experiment of the invention in a file format suitable for retrieval and processing in a computerized analysis, comparison, or relative quantitation method, for example.

The invention also provides a network, comprising a plurality of computing devices linked via a data link, such as an Ethernet cable (coax or 10 BaseT), telephone line, ISDN line, wireless network, optical fiber, or other suitable signal transmission medium, whereby at least one network device (e.g., computer, disk array, etc.) comprises a pattern of magnetic domains (e.g., magnetic disk) and/or charge domains (e.g., an array of DRAM cells) composing a bit pattern encoding data acquired from an assay of the invention.

The invention also provides a method for transmitting assay data that includes generating an electronic signal on an electronic communications device, such as a modem, ISDN terminal adapter, DSL, cable modem, ATM switch, or the like, wherein the signal includes (in native or encrypted format) a bit pattern encoding data from an NMR assay or a database comprising a plurality of NMR results obtained by the method of the invention.

In a preferred embodiment, the invention provides a computer system for comparing a query target to a database containing an array of data structures, such as a NMR assay result obtained by the method of the invention, and ranking database targets based on the degree of identity and gap weight to the target data. A central processor is preferably initialized to load and execute the computer program for alignment and/or comparison of the assay results. Data for a query target is entered into the central processor via an I/O device. Execution of the computer program results in the central processor retrieving the assay data from the data file, which comprises a binary description of a NMR assay result.

The target data or record and the computer program can be transferred to secondary memory, which is typically random access memory (e.g., DRAM, SRAM, SGRAM, or SDRAM). Targets are ranked according to the degree of correspondence between a selected assay characteristic (e.g., binding to a selected affinity moiety) and the same characteristic of the query target and results are output via an I/O device. For example, a central processor can be a conventional computer (e.g., Intel Pentium, PowerPC, Alpha, PA-8000, SPARC, MIPS 4400, MIPS 10000, VAX, etc.); a program can be a commercial or public domain molecular biology software package (e.g., UWGCG Sequence Analysis Software, Darwin); a data file can be an optical or magnetic disk, a data server, a memory device (e.g., DRAM, SRAM, SGRAM, SDRAM, EPROM, bubble memory, flash memory, etc.); an I/O device can be a terminal comprising a video display and a keyboard, a modem, an ISDN terminal adapter, an Ethernet port, a punched card reader, a magnetic strip reader, or other suitable I/O device.

The invention also preferably provides the use of a computer system, such as that described above, which comprises: (1) a computer; (2) a stored bit pattern encoding a collection of peptide sequence specificity records obtained by the methods of the invention, which may be stored in the computer; (3) a comparison target, such as a query target; and (4) a program for alignment and comparison, typically with rank-ordering of comparison results on the basis of computed similarity values.

The materials, methods and devices of the present invention are farther illustrated by the examples that follow. These examples are offered to illustrate, but not to limit the claimed invention.

### EXAMPLES

Example 1 set forth the procedure for preparing cells having a selected macromolecule that was labeled with an NMR-detectable nucleus. Example 2 illustrated an embodiment of the NMR technique of the invention.

Examples 3 through 7 investigated the influence on the NMR spectra of the selected macromolecule of varying the cell culture conditions.

### Example 1

1.1 Protein Overexpression

NmerA is the N-terminal domain of the bacterial detoxification protein MerA that accumulates in the bacterial cytoplasm to levels of up to 6% of total soluble protein in response to mercurials (Misra et al., Gene 1985, 34, 253-262; Fox et al., J. Biol. Chem. 1982, 257, 2498-2503; and Miller, S. M., Essays in Biochemistry, 34:17-30 (1999)). The N-terminal metal binding domain of MerA containing amino acids 1-69 was cloned into a pET-11a vector (Stratagene) by standard PCR techniques. BL21 DE3 E. coli bacteria were transformed with the plasmid and selected for transformation on an ampicillin plate. The cells were grown in different media at 37° C. in a rotary shaker. Unless stated otherwise, cells were first grown in LB medium to an optical density of 1.2 and harvested by centrifugation at 850 g for 20 minutes. The pellet was then resuspended in different media and induced with 0.4 mM IPTG. Four hours post-induction, the bacteria were harvested by gentle centrifugation (170 g for 25 minutes), which formed an easily dislodged, poorly packed pellet at the bottom of a conical tube. Samples that were selectively labeled with ¹⁵N on lysines were produced by expressing the protein in minimal medium containing 100 mg per liter of the labeled amino acid.

### Example 2

2.1 NMR Spectroscopy

All NMR experiments were measured on a Bruker 500 MHz Avance NMR instrument equipped with a triple resonance cryoprobe. Due to the insensitivity of the bacterial sample to shimming, a separate sample of the same height containing the supernatant of the harvested cells was used to shim. All HSQC experiments were measured at 37° C. with a standard FHSQC pulse sequence employing WATERGATE for water suppression. In the ¹H acquisition dimension, 1024 complex data points with a t₂max of 80 ms were recorded. In the indirect ¹⁵N-dimension, 60 complex points with a t₁max of 41 ms were measured. Unless stated otherwise, all spectra were collected with four scans per increment. The total measurement time per experiment was less than ten minutes. All spectra were transformed using the XWINNMR software package (Bruker). A wide-bore glass pipette was used to suck the bacterial pellet from the bottom and to place 460 μl into a 5 mm NMR tube already containing 40 μl of deuterium oxide. A small air bubble was included in the bacterial slurry to mix and homogenize the sample by carefully inverting the tube back-and-forth.

### Example 3

3.1 The Effect of the Polymerase Inhibitor Rifampicin on Background Signals

To minimize the ¹⁵N incorporation into proteins and cellular molecules other than the selected macromolecule, a two-step protocol was used. Cells harboring the expression plasmid were first grown in unlabeled LB medium. After harvesting by centrifugation, they were resuspended in ¹⁵N-labeled minimal medium. Ten minutes after resuspension, the cells were induced with 0.4 mM isopropyl β-D-thiogalactopyranoside (IPTG). Forty minutes after induction, the RNA polymerase inhibitor rifampicin was added to the bacterial culture to a concentration of 35 μM. Rifampicin suppresses the production of all bacterial proteins, while the protein of interest, NmerA, is under the control of a T7 promoter. The polymerase of the bacteriophage T7 was not affected by the drug, which enables the selective expression of a single protein in bacteria (Sippel et al., Biochimica et Biophysica Acta, 157:218-219 (1968); Richardson et al,. In Escherichia coli and Salmonella; Neidhardt, F. C., Ed.; ASM Press: Washington, D.C., 1996; Vol. 1, pp 822-848; Campbell et al., Cell, 104:901-912 (2001).

3.2 Results

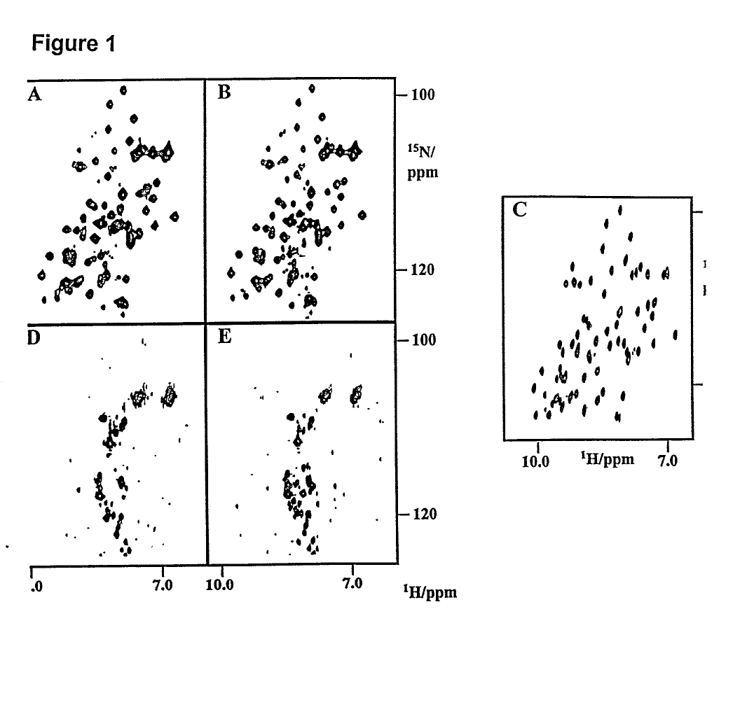

To evaluate the effect of suppressing the bacterial protein production by rifampicin, NmerA was expressed in the presence and in the absence of the drug while leaving all other parameters unchanged. The two HSQC experiments obtained with the in-cell NmerA samples expressed in the absence and presence of rifampicin are shown in FIG. 1A and FIG. 1B, respectively. In addition, an in vitro HSQC spectrum of purified NmerA is shown in FIG. 1C.

Comparison of the three spectra in FIG. 1A to FIG. 1C showed that they were very similar. Both in-cell HSQC (FIG. 1A and FIG. 1B) spectra contained, in addition to the protein resonances of NmerA, several sharp NMR signals in the range of 8-8.5 ppm. The sharpness of these lines suggested that they did not originate from protein signals but from the incorporation of ¹⁵N into small molecules like amino acids. Interestingly, both spectra contained the same artifacts but did not show any signs of additional protein resonances. This result suggested that rifampicin was not necessary to suppress potential NMR signals of bacterial proteins. To further investigate the influence of rifampicin on the ¹⁵N-incorporation into small organic molecules, two samples were produced as described above. However, this time the bacterial samples were not induced. The resulting HSQC spectra of these non-induced samples are shown in FIG. 1D for a sample without rifampicin and in FIG. 1E for a sample containing rifampicin. Like the spectra of the induced samples, both spectra were very similar with even a slight increase in the number of NMR signals in the rifampicin sample, suggesting that addition of rifampicin to bacterial samples did not have any effect on the suppression of background NMR signals in in-cell NMR experiments.

### Example 4

4.1 Bacterial Growth and Protein Expression Phase Medium Switch

The influence of switching the medium from unlabeled LB medium to ¹⁵N-labeled minimal medium prior to induction was investigated. Three different protocols were used to produce in-cell NMR samples of NmerA.

4.1a The bacteria were grown in ¹⁵N-labeled minimal medium to an optical density of 0.8. The expression of NmerA was induced by addition of IPTG in the same medium.

4.1b The bacteria were grown in ¹⁵N-labeled minimal medium to an optical density of 0.8. The bacteria were harvested by centrifugation at 850 g and resuspended in fresh ¹⁵N-labeled minimal medium before induction with IPTG.

4.1c The bacteria were grown in LB medium and harvested by centrifugation. The bacteria were resuspended in ¹⁵N-labeled minimal medium to the same optical density as the sample in 4.1b.

4.2 Results

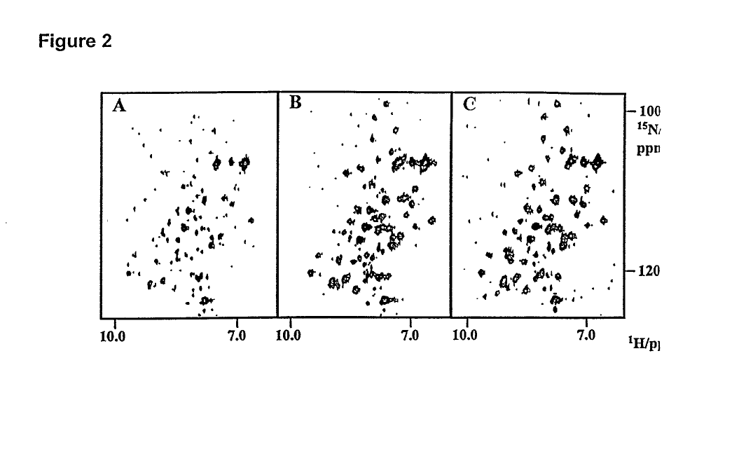

The resulting HSQC spectra of samples 4.1a to 4.1c are shown in FIG. 2. All three spectra showed a very similar level of background signals, suggesting that switching the type of medium prior to induction had a negligible effect on the suppression of these signals. The spectra, however, showed large differences in the intensity of the protein peaks. The sample obtained by growing and expressing the protein in the same minimal medium clearly exhibited the lowest sensitivity. Switching the medium to fresh ¹⁵N-labeled minimal medium prior to induction increased the spectral quality several fold. The type of medium used to grow the bacteria in the first phase before induction seemed to have had only a very small influence on the resulting spectrum, with the sample that was initially grown in LB medium showing a slightly higher sensitivity than the spectrum that was grown in minimal medium.

### Example 5

5.1 Investigation of the Influence of the Overexpression Level

The lower limit for the observation of overexpressed proteins inside living bacteria was investigated by inducing NmerA for varying amounts of time. The spectra were measured with 4 scans per increment, as described above, and establish the lower detection limit for in-cell NMR experiments.

5.2 Results

The combined results of the rifampicin experiments and the studies of changing the media suggested that the amount of background signal arising from ¹⁵N incorporation into cellular components other than selected macromolecule is small and is insensitive to the specific growth and induction protocol used. This implied that the behavior of the individual protein was an important factor for observing proteins inside living bacterial cells.

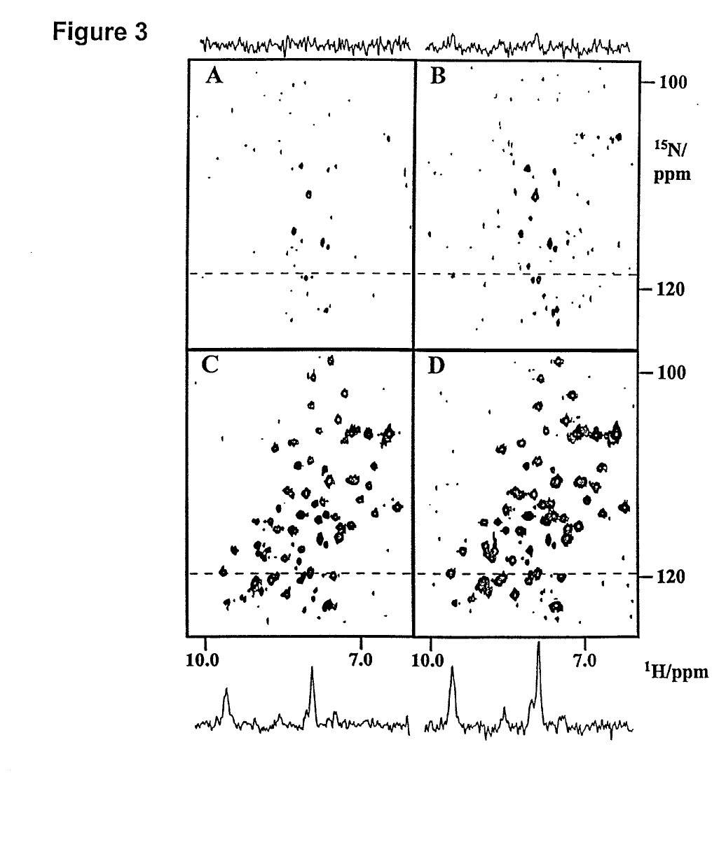

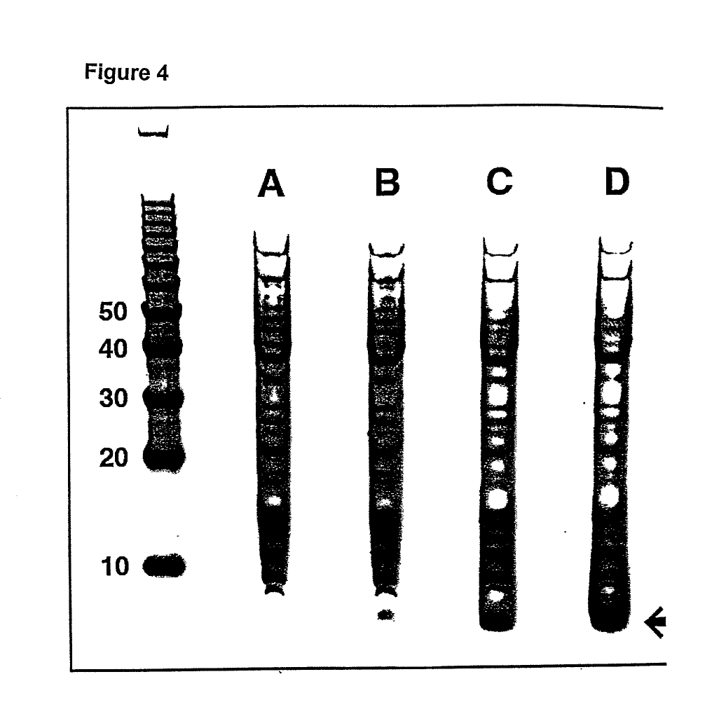

The resulting spectra are shown in FIG. 3. FIG. 4 shows a gel that demonstrated the level of NmerA overexpression that corresponded to the spectra in FIG. 3. Ten minutes after induction the in-cell HSQC showed only some background signals (FIG. 3A) and NmerA could not be detected on the gel. After 30 minutes some weak protein resonances became visible in the HSQC spectrum (FIG. 3B) and a faint band of NmerA appeared. One hour post-induction all resonances seen in in-cell NMR experiments of NmerA were visible, and after two hours the signals became stronger. The corresponding gel lanes showed a strong NmerA band. For a better comparison of the signal-to-noise ratios, one-dimensional cross-sections along the acquisition dimension taken at the position of the dotted line were shown for each spectrum.

Although the intensity of the bands in the spectrum shown in FIG. 3B was only approximately related to the intra-cellular concentration of the protein, it was estimated from the NmerA band in lane B in FIG. 4 that the detection limit for a protein in in-cell NMR experiments was only a few percent of the total amount of soluble protein. Furthermore, it was estimated from these data that an approximately 5% overexpression level was sufficient to provide high quality in-cell NMR spectra.

### Example 6

6.1 Improvement of Spectral Quality by Expression in Labeled Rich Media

To investigate if the quality of the spectra could be enhanced by expressing the protein in rich, labeled media, the bacteria were grown in LB medium to an optical density of 1.2. The bacteria were harvested by centrifugation, half of the pellet was resuspended in standard ¹⁵N-labeled minimal medium and the other half in ¹⁵N-labeled rich medium. This rich medium was produced from 13.3 g/L of 98% ¹⁵N-labeled and 97% deuterated algae extract (Celtone-dN, Martek) dissolved in H₂O. Overexpressing proteins in bacteria grown in deuterated media dissolved in H₂O has been shown to give approximately 80% deuteration on methyl groups and 50% deuteration on the α-protons leading to a twofold reduction of the proton T₂ relaxation rate (Markus et al., J. Magn. Reson. B, 105:192-195 (1994)).

6.2 Results

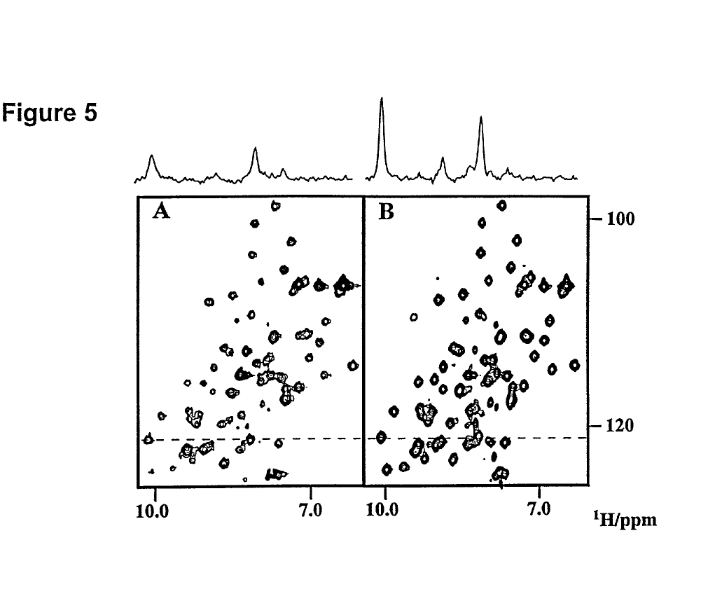

The HSQC spectra of the in-cell samples are shown in FIG. 5. The spectrum of NmerA expressed in the rich medium clearly showed a two- to three-fold higher sensitivity. This higher sensitivity could be attributed both to the higher protein expression level in the rich medium as well as to the effect of the deuteration. The comparison of one-dimensional cross section through peaks of the HSQC spectra showed a reduction in the amide proton line width from an average of 55 Hz in the non-deuterated sample to 40 Hz in the partially deuterated sample.

### Example 7

7.1 Selective Amino Acid Labeling.

NmerA expressed as discussed above in standard, unlabeled minimal medium that was supplemented with 0.1 g/l of ¹⁵N-labeled lysine (CIL). A modification of the above-described method was used to prepare ¹⁵N-labeled calmodulin.

7.2 Results

In-cell NMR spectra can have greater peak overlap relative to in vitro spectra due to larger linewidths. One potential method to improve resolution is selective ¹⁵N-labeling of only certain types of amino acids (Waugh D. S., J. Biomol. NMR, 8:184-192 (1996)). This method is particularly powerful if only a certain type of amino acid is of interest, e.g. a residue in the active site of an enzyme.

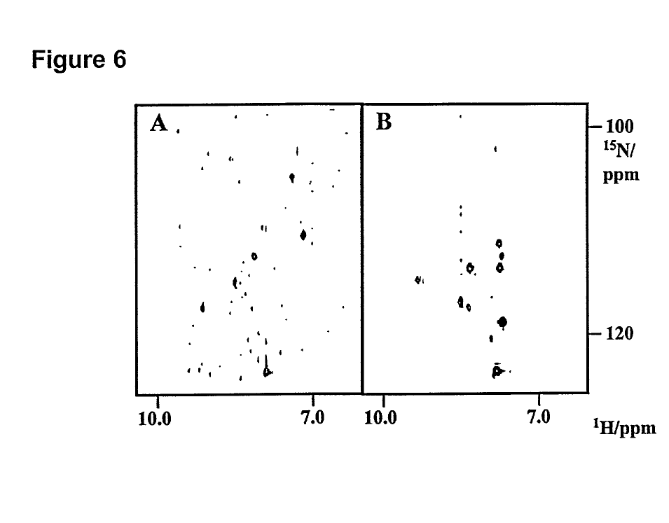

FIG. 6 A shows an in-cell HSQC experiment of NmerA expressed in standard, unlabeled minimal medium that was supplemented with 0.1 g/l of ¹⁵N-labeled lysine (CIL). The spectrum contained six peaks, five of which corresponded to the five lysines of NmerA. The sixth and by far strongest peak represented a metabolic product of ¹⁵N-labeled lysine.

As a second example, FIG. 6 B showed an in-cell HSQC spectrum of human calmodulin selectively labeled on lysines. In addition to the expected 7 resonances, some minor peaks were visible, which might represent protein species with different metal ions in the four binding sites.

The above-described experiments demonstrated that selective amino acid labeling and selective observation of certain types of amino acids in living cells was possible without any background signal with the exception of a metabolic product of lysine. Not all types of amino acids, however, are good candidates for selective ¹⁵N-labeling in E. coli BL21 cells. Some amino acids are precursors for other amino acids, and aminotransferases can transfer (¹⁵N-labeled) amino groups between amino acid types (Waugh, supra). Lysine as well as other end products of biosynthetic pathways in E. coli,however, can be used. Selective labeling of other amino acid types can be facilitated by expression of a protein of interest in special E. coli strains that are auxotrophic for a particular amino acid and exogenously supplying the labeled amino acid to the E. coli.

It is understood that the examples and embodiments described herein were for illustrative purposes only and that various modifications or changes in light thereof will be suggested to persons skilled in the art and are to be included within the spirit and purview of this application and are considered within the scope of the appended claims. All publications, patents, and patent applications cited herein are hereby incorporated by reference in their entirety for all purposes.

What is claimed is:

1. A method of extracting structural information from a NMR data set for a selected macromolecule in an intact biological compartment wherein said selected macromolecule is labeled with an NMR-detectable nucleus, such that said nucleus is present in said macromolecule in an amount greater than is naturally abundant in said macromolecule, said method comprising:
(a) contacting said cell with radio frequency energy, thereby producing an excited NMR-detectable nucleus;
(b) collecting radio frequency data from said excited NMR-detectable nucleus, thereby producing said NMR data set, and
(c) analyzing said data set to extract said structural information for said selected macromolecule from said data set.

2. The method according to claim 1, wherein said selected macromolecule is overexpressed in said biological compartment.

3. The method according to claim 1, wherein said NMR-detectable nucleus is present in an amount detectable by NMR of said biological compartment.

4. The method according to claim 1, wherein said selected macromolecule is a member selected from the group consisting of proteins, saccharides, glycoproteins, and nucleic acids.

5. The method according to claim 1, wherein said selected macromolecule is in a complex with a small molecule.

6. The method according to claim 5, wherein said small molecule is an exogenous small molecule.

7. The method according to claim 5, wherein said small molecule is a therapeutic agent or a candidate therapeutic agent.

8. The method according to claim 7, wherein said small molecule is an exogenous small molecule.

9. The method according to claim 1, wherein said macromolecule is further labeled with deuterium.

10. The method according to claim 1, wherein said biological compartment is present in a suspension.

11. The method according to claim 1, wherein said structural information is conformational information.

12. The method according to claim 1, wherein said structural information is for a complex formed between said selected macromolecule and a small molecule selected from therapeutic agents and candidate therapeutic agents.

13. The method according to claim 1, wherein said structural information is for a complex formed between said selected macromolecule and a member selected from small molecules, endogenous macromolecules and combinations thereof.

14. The method according to claim 1, wherein said structural information is for a first conformation of said selected macromolecule and a second conformation of said selected macromolecule.

15. The method according to claim 1, wherein said data set is acquired by a triple resonance NMR method.

16. The method according to claim 15, wherein said triple resonance NMR experiment is a member selected from HSQC and TROSY.

17. The method according to claim 1, wherein said biological compartment is prepared by a method comprising:
(a) transforming an unlabeled precursor of said labeled biological compartment with a nucleic acid encoding said selected macromolecule, wherein said nucleic acid is operably linked to a promoter non-native to said unlabeled precursor cell, thereby producing a transformed biological compartment;
(b) incubating said transformed biological compartment in a medium comprising said NMR-detectable nucleus; and
(c) inducing said transformed biological compartment, thereby preparing said labeled biological compartment.

18. The method according to claim 17, further comprising:
(d) inhibiting essentially all transcription in said transformed biological compartment, which is under control of promoters native to said unlabeled precursor biological compartment, while allowing transcription under control of said non-native promoter to proceed.

19. The method according to claim 17, wherein said medium comprises an amino acid labeled with said NMR sensitive nucleus.

20. The method according to claim 17, wherein said medium is deuterated.

21. The method according to claim 17, wherein said biological compartment is a bacterial cell.

22. The method according to claim 17, wherein the non-native promoter encodes an RNA polymerase that is operable during step (d).

23. The method according to claim 17, wherein the non-native promoter is a phage promoter.

24. The method according to claim 18, wherein said inhibiting is caused by administering an inhibitor to said biological compartment in an amount sufficient to cause said inhibiting.

25. The method according to claim 24, wherein said inhibitor is rifampicin.

26. The method of claim 1, wherein said selected macromolecule experiences a local viscosity at least 2 fold greater than the viscosity of pure water, wherein said local viscosity and said viscosity of said pure water are determined at the same temperature.

27. The method of claim 1, wherein said selected macromolecule is present in said biological compartment at a weight percent of up to 0.3% compared to the total weight of said biological compartment.

28. The method of claim 1, wherein said selected macromolecule is present in said biological compartment at a weight percent of up to 50% compared to the total weight of said biological compartment.

29. The method of claim 1, wherein said selected macromolecule has a molecular weight of at least 5 kDa.

30. The method of claim 1, wherein said selected macromolecule has a molecular weight of at least 25 kDa.

31. The method of claim 1, wherein said selected macromolecule has a molecular weight of at least 70 kDa.

32. The method of claim 1, wherein said biological compartment is a living cell.

33. The method of claim 1, wherein said biological compartment is a cell that has been metabolically arrested.

34. The method of claim 1, wherein said selected macromolecule is expressed from a plasmid.

35. The method of claim 1, using a multidimensional multinuclear method.

36. The method of claim 35, using an HNCA experiment.

37. The method of claim 35, using an HMQC experiment.

38. The method of claim 1, wherein said compartment is a biological cell.

39. The method of claim 38, wherein said cell is a prokaryotic cell.

40. The method of claim 39, wherein said cell is a E. coli cell.

41. The method of claim 38, wherein said cell is a eukaryotic cell.

42. The method of claim 41, wherein said cell is a yeast cell.

43. The method of claim 41, wherein said cell is a mammalian cell.

44. The method of claim 43, wherein said cell is a human cell.

45. A method of extracting structural information from a NMR data set for a selected macromolecule of an intact biological compartment wherein said selected macromolecule is labeled with a NMR-detectable nucleus, such that said nucleus is present in said macromolecule in an amount greater than is naturally abundant in said macromolecule, wherein said nucleus is not ¹⁹F, said method comprising:
(a) contacting said biological compartment with radio frequency energy, thereby producing an excited NMR-detectable nucleus, and
(b) collecting radio frequency data from said excited NMR-detectable nucleus, thereby producing said NMR data set.

46. The method according to claim 45, wherein said selected macromolecule is overexpressed in said biological compartment.

47. The method according to claim 45, wherein said NMR-detectable nucleus is present in an amount detectable by NMR of said intact, biological compartment.

48. The method according to claim 45, wherein said selected macromolecule is a member selected from the group consisting of proteins, saccharides, glycoproteins, and nucleic acids.

49. The method according to claim 45, wherein said selected macromolecule is in a complex with a small molecule.

50. The method according to claim 49, wherein said small molecule is an exogenous small molecule.

51. The method according to claim 49, wherein said small molecule is a therapeutic agent or a candidate therapeutic agent.

52. The method according to claim 51, wherein said small molecule is an exogenous small molecule.

53. The method according to claim 45, wherein said macromolecule is further labeled with deuterium.

54. The method according to claim 45, wherein said biological compartment is present in a suspension.

55. The method according to claim 45, wherein said structural information is conformational information.

56. The method according to claim 45, wherein said structural information is for a complex formed between said selected macromolecule and a small molecule selected from therapeutic agents and candidate therapeutic agents.

57. The method according to claim 45, wherein said structural information is for a complex formed between said selected macromolecule and a member selected from small molecules, endogenous macromolecules and combinations thereof.

58. The method according to claim 45, wherein said structural information is for a first conformation of said selected macromolecule and a second conformation of said selected macromolecule.

59. The method according to claim 45, wherein said data set is acquired by a triple resonance NMR method.

60. The method according to claim 59, wherein said triple resonance NMR experiment is a member selected from HSQC and TROSY.

61. The method according to claim 45, wherein said biological compartment is prepared by a method comprising:
(a) transforming an unlabeled precursor of said labeled biological compartment with a nucleic acid encoding said selected macromolecule, wherein said nucleic acid is operably linked to a promoter non-native to said unlabeled precursor biological compartment, thereby producing a transformed biological compartment;
(b) incubating said transformed biological compartment in a medium comprising said NMR-detectable nucleus; and
(c) inducing said transformed biological compartment, thereby preparing said labeled biological compartment.

62. The method according to claim 61, further comprising:
(d) inhibiting essentially all transcription in said transformed biological compartment, which is under control of promoters native to said unlabeled precursor biological compartment, while allowing transcription under control of said non-native promoter to proceed.

63. The method according to claim 61, wherein said medium comprises an amino acid labeled with said NMR sensitive nucleus.

64. The method according to claim 61, wherein said medium is deuterated.

65. The method according to claim 61, wherein said biological compartment is a bacterial cell.

66. The method according to claim 61, wherein the non-native promoter encodes an RNA polymerase that is operable during step (d).

67. The method according to claim 61, wherein the non-native promoter is a phage promoter.

68. The method according to claim 62, wherein said inhibiting is caused by administering an inhibitor to said biological compartment in an amount sufficient to cause said inhibiting.

69. The method according to claim 68, wherein said inhibitor is rifampicin.

70. The method of claim 45, wherein said selected macromolecule experiences a local viscosity at least 2 fold greater than the viscosity of pure water, wherein said local viscosity and said viscosity of said pure water are determined at the same temperature.

71. The method of claim 45, wherein said selected macromolecule is present in said biological compartment at a weight percent of up to 0.3% compared to the total weight of said biological compartment.

72. The method of claim 45, wherein said selected macromolecule is present in said biological compartment at a weight percent of up to 50% compared to the total weight of said biological compartment.

73. The method of claim 45, wherein said selected macromolecule has a molecular weight of at least 5 kDa.

74. The method of claim 45, wherein said selected macromolecule has a molecular weight of at least 25 kDa.

75. The method of claim 45, wherein said selected macromolecule has a molecular weight of at least 70 kDa.

76. The method of claim 45, wherein said biological compartment is a living cell.

77. The method of claim 45, wherein said biological compartment is a cell that has been metabolically arrested.

78. The method of claim 45, wherein said selected macromolecule is expressed from a plasmid.

79. The method of claim 45, using a multidimensional multinuclear method.

80. The method of claim 79, using an HNCA experiment.

81. The method of claim 79, using an HMQC experiment.

82. The method of claim 45, wherein said compartment is a biological cell.

83. The method of claim 82, wherein said cell is a prokaryotic cell.

84. The method of claim 83, wherein said cell is a E. coli cell.

85. The method of claim 83, wherein said cell is a eukaryotic cell.

86. The method of claim 85, wherein said cell is a yeast cell.

87. The method of claim 85, wherein said e cell is a mammalian cell.

88. The method of claim 87, wherein said cell is a human cell.
